(19) United States
(12) Patent Application Publication
(10) Pub. No.: US 2025/0029245 A1
(43) Pub. Date: Jan. 23, 2025

(54) HUMAN-MACHINE INTERACTION FOR OPERATING A MEDICAL APPARATUS

(71) Applicant: Siemens Healthineers AG, Forchheim (DE)

(72) Inventor: Amilcar ALZAGA, Schoenbrun Im Steigerwald (DE)

(73) Assignee: Siemens Healthineers AG, Forchheim (DE)

(21) Appl. No.: 18/778,082

(22) Filed: Jul. 19, 2024

(30) Foreign Application Priority Data
Jul. 20, 2023 (EP) 23186781.3

(51) Int. Cl. G06T 7/00; G16H 30/40

(52) U.S. Cl. CPC G06T 7/0012; G16H 30/40; G06T 2207/20084

(57) ABSTRACT

In a method for human-machine interaction for operating a medical apparatus, a first user input is captured, wherein the first user input specifies an intention to operate the medical apparatus for a usage case. Depending on the medical apparatus and the usage case, at least one input suggestion is generated for a computer-implemented language model, and a representation of the at least one input suggestion is output. A second user input is captured, wherein the second user input validates or rejects a first input suggestion of the at least one input suggestion. In response to the first input suggestion being validated by the second user input, user information for controlling the medical apparatus for the usage case is generated and output by applying the computer-implemented language model to the first input suggestion.

### CROSS-REFERENCE TO RELATED APPLICATION(S)

The present application claims priority under 35 U.S.C. § 119 to European Patent Application No. 23186781.3, filed Jul. 20, 2023, the entire contents of which is incorporated herein by reference.

### FIELD

One or more embodiments of the present invention relate to a computer-implemented method for human-machine interaction for operating a medical apparatus, to a data processing apparatus for performing said computer-implemented method, to a corresponding non-transitory computer program product, and to a non-transitory computer-readable storage medium.

### BACKGROUND

Medical apparatuses, for example imaging modalities such as ultrasound imaging systems, X-ray based imaging systems and magnetic resonance tomography systems, or apparatuses for carrying out therapeutic methods, for instance HIFU systems (HIFU stands for “high-intensity focused ultrasound”) or histotripsy systems, are often highly complex and can have a large number of functions.

To be able to use such medical apparatuses appropriately often requires not only basic medical training and training specific to the particular medical apparatus, but also a large amount of practice and hands-on experience. In addition, it may be necessary to keep the respective knowledge of the work processes and functions constantly up to date.

Consequently, this can lead to incorrect operation of medical apparatuses and/or a failure to make full use of their functionality.

Language models, in particular what are known as large language models (LLMs), for instance the GPT-4 model described in the publication “GPT-4 Technical Report” (arXiv: 2303.08774v3), can be used as virtual assistants. A problem here are irrelevant statements or what are known as hallucinated responses or statements, which are not justified by the underlying training data.

### SUMMARY

An object of one or more embodiments of the present invention is to define a method for human-machine interaction for operating a medical apparatus, in which method a user can be assisted more reliably.

At least this object is achieved by the subject matter of the independent claim. The subject matter of the dependent claims contains further developments and preferred embodiments.

Independent of the grammatical term usage, individuals with male, female or other gender identities are included within the term.

According to one aspect of embodiments of the present invention, a computer-implemented method is defined for human-machine interaction for operating a medical apparatus. In this method, a first user input is captured, which specifies an intention of a user to operate the medical apparatus for a usage case. Depending on the medical apparatus and the usage case, at least one input suggestion is generated for a computer-implemented language model, and a representation of the at least one input suggestion is output. In response to the output of the representation of the at least one input suggestion, i.e. in particular after the output of the representation of the at least one input suggestion, a second user input is captured, which validates a first input suggestion of the at least one input suggestion or rejects the at least one input suggestion, so in particular rejects all the input suggestions of the at least one input suggestion. If the first input suggestion was validated by the second user input, user information for controlling the medical apparatus for the usage case is generated and output by applying the language model to the first input suggestion.

Unless stated otherwise, all the steps of the computer-implemented method can be performed by a data processing apparatus that has at least one computing unit. In particular, the at least one computing unit is configured or adapted to perform the steps of the computer-implemented method. For this purpose, the at least one computing unit can store, for example, a computer program containing commands which, on execution by the at least one computing unit, cause the at least one computing unit to execute the computer-implemented method.

A medical apparatus can be any technical apparatus for carrying out, in particular at least in part automatically, a diagnostic, therapeutic, surgical and/or imaging method. The carrying out of a diagnostic, therapeutic and/or surgical imaging method, however, cannot be generally considered to be part of the computer-implemented method according to embodiments of the present invention. Instead, the user information provided according to embodiments of the present invention can be used to carry out such a method.

The computer-implemented language model is in particular what is known as a large language model, LLM. An input suggestion can be understood to mean a suggestion for an input, also referred to as a prompt, to the language model.

The language model can be provided in particular in trained form, i.e. can be a known language model. It is also possible that the language model is provided in pre-trained form, and is refined on the basis of further training data, for example via a suitably provided application programming interface (API).

A user input can be made, for example, in text form or in voice form, for example followed by voice-to-text conversion. The representation of the at least one input suggestion contains a representation of the first input suggestion and in particular, if the at least one input suggestion contains two or more input suggestions, a representation of each of all the input suggestions of the at least one input suggestion. A representation of an input suggestion can be a visual representation, for example, in particular a representation in text form. The output of the representation of the at least one input suggestion can be made, for example, visually on a display device and/or acoustically by a suitable loudspeaker system.

The second user input validating the first input suggestion can be understood to mean that the user expresses that he approves of the first input suggestion, on the basis of a content of the second user input.

The second user input rejecting the at least one input suggestion can be understood to mean that the user expresses that he does not approve of any input suggestion of the at least one input suggestion, on the basis of a content of the second user input. The content of the second user input can be limited to this, or can contain further information, for instance a reason for the rejection or an improvement suggestion, and so on.

The output of the user information for the at least one input suggestion can be made, for example, visually on a display device and/or acoustically by the loudspeaker system.

The first user input specifies, for example, the particular medical apparatus and the particular usage case to which the user wants to apply the medical apparatus. The particular medical apparatus can be chosen, for example, from a multiplicity of predefined medical apparatuses. Furthermore, in some embodiments, the first user input can also contain further information, for instance relating to a user's training, a level of knowledge of the user, a composition of a team of people for carrying out the particular use, personal or anatomical or pathological information about a patient, symptoms or a history of disease of the patient, results from earlier medical examinations, and so on.

As a result of the at least two-stage procedure, namely the providing of the at least one input suggestion in response to the first user input, and the applying of the language model only if an input suggestion is validated, the user information can be provided more reliably.

According to at least one embodiment, the user information contains system settings for the medical apparatus.

Based on the user information, the user can thus set up the medical apparatus correctly.

According to at least one embodiment, if the first input suggestion was validated by the second user input, a parameter, for instance the system settings, of the medical apparatus is set automatically on the basis of the user information.

According to at least one embodiment, if the first input suggestion was validated by the second user input, the medical apparatus is controlled automatically on the basis of the user information in order to perform a measurement automatically.

According to at least one embodiment, the user information contains guidance for operating the medical apparatus for the usage case, in particular in text form.

The user can be guided thereby step by step to perform the necessary operating steps.

According to at least one embodiment, the user information contains one or more pictorial representations of a procedure for operating the medical apparatus for the usage case.

This can assist the user visually in the operation of the medical apparatus.

According to at least one embodiment, the language model contains a trained artificial neural network.

Artificial neural networks have proven to be extremely powerful algorithms, especially for large language models.

According to at least one embodiment, the artificial neural network is in the form of a generative pre-trained transformer (GPT).

Such neural networks, for instance the GPT-4 mentioned in the introduction, have proven to be particularly powerful.

According to at least one embodiment, the at least one input suggestion contains the first input suggestion and a second input suggestion. The second user input either selects and validates the first input suggestion or rejects the at least one input suggestion, so in particular rejects both the first input suggestion and the second input suggestion.

The at least one input suggestion thus contains at least two input suggestions, preferably three or more, from which the user can choose. Hence the user can select in particular that input suggestion that comes closest to his intention. In this way, the quality of the user information can be improved, or unnecessary repetitions or corrections can be avoided.

According to at least one embodiment, source information relating to the user information is output.

The source information is in particular information that states one or more sources that were used by the language model as the basis for producing the user information. The sources are in particular part of the training data that was used to train the language model.

Thus the user can thereby check the plausibility of, or verify, the user information.

According to at least one embodiment, the source information contains a textual and/or pictorial representation of at least one extract from an information source for the user information, and the textual and/or pictorial representation is displayed on a display device, wherein a part that is relevant to the user information is highlighted visually.

In other words, the source information not only states the information source from which a relevant part of the user information originates, but also highlights visually precisely which part, in particular which text passage or text passage and/or which illustration or illustrations, of the information source forms the basis of the relevant part of the user information. This further simplifies the plausibility check or verification of the user information.

The information source may be, for example, a scientific publication, a textbook, a work instruction and so on.

According to at least one embodiment, if the at least one input suggestion was rejected by the second user input, depending on the medical apparatus and the usage case, at least one further input suggestion is generated for the language model, and a representation of the at least one further input suggestion is output. In response thereto, a third user input is captured, which validates a first further input suggestion of the at least one further input suggestion or rejects the at least one further input suggestion. If the first further input suggestion was validated by the third user input, further user information for controlling the medical apparatus for the usage case is generated and output by applying the language model to the first further input suggestion.

Hence the quality of the user information or of the further user information can be improved further. An analogous iterative procedure can be followed until a validated input suggestion exists. It is also possible, however, that the method is terminated if no validated input suggestion exists after a predefined number of iterations.

According to at least one embodiment, the at least one further input suggestion is generated according to content of the second user input.

In other words, the second user input contains not only the information that the at least one input suggestion is rejected, but also more extensive information about how, from the user's point of view, an improved generated input suggestion would be achievable. This more extensive information can then be taken into account in producing the at least one further input suggestion.

According to an optional embodiment, a fourth user input is captured. The fourth user input is captured in particular after the generation and/or output of the user information. The fourth user input specifies an acceptance and/or applicability of the user information for a usage case. The user can specify by way of the fourth user input whether the user information is, and/or appears to be, usable, applicable and/or appropriate for the usage case, the patient, the medical apparatus. In particular, the user can enter and/or provide his approval and/or his distrust of the user information via the fourth user input. The acceptance can specify, for example, to what extent the user trusts the correctness and/or appropriateness of the user information. If the fourth user input specifies that the user information is not acceptable, applicable, usable, appropriate and/or trustworthy, then further, in particular improved and/or iterative, user information is generated and/or output. The further, improved and/or iterative, user information is generated and/or determined by applying the language model to the first input suggestion and the fourth user input.

According to at least one embodiment, the medical apparatus is an imaging modality, in particular an X-ray imaging system, a magnetic resonance tomography system, or an ultrasound-based imaging system.

For usage cases or usage situations that can arise in a method according to embodiments of the present invention and are not explicitly described here, it can be provided that, according to the method, an error message and/or a request to enter user feedback is output and/or a default setting and/or a predetermined initial state is set.

According to a further aspect of embodiments of the present invention, a data processing apparatus having at least one computing unit is defined. The at least one computing unit is configured to perform a computer-implemented method according to embodiments of the present invention.

A computing unit can be understood to mean in particular a data processing unit which contains a processing circuit. In particular, the computing unit can thus process data for performing computing operations. These include also operations for performing indexed accesses to a data structure, for instance to a look-up table (LUT).

The computing unit can contain in particular one or more computers, one or more microcontrollers, and/or one or more integrated circuits, for example one or more application-specific integrated circuits (ASIC), one or more field-programmable gate arrays (FPGA), and/or one or more systems on a chip (SoC). The computing unit can also contain one or more processors, for example one or more microprocessors, one or more central processing units (CPU), one or more graphics processing units (GPU), and/or one or more signal processors, in particular one or more digital signal processors (DSP). The computing unit can also contain a physical or virtual interconnection of computers or other of the aforementioned units.

In various exemplary embodiments, the computing unit contains one or more hardware and/or software interfaces and/or one or more memory units.

A memory unit can be embodied as a volatile data storage medium, for example as a dynamic random access memory (DRAM) or a static random access memory (SRAM), or as a non-volatile data storage medium, for example as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM), or as a phase-change random access memory (PCRAM).

According to a further aspect of embodiments of the present invention, a computer program containing commands is defined. When the commands are executed by a data processing apparatus, in particular by a data processing apparatus according to embodiments of the present invention, the commands cause the data processing apparatus to perform a computer-implemented method according to embodiments of the present invention.

For example, the commands can exist as program code. The program code can be provided, for example, as binary code or assembler and/or as source code of a programming language, for instance C, and/or as program script, for instance Python.

According to a further aspect of embodiments of the present invention, a non-transitory computer-readable storage medium is defined, which stores a computer program according to embodiments of the present invention.

The computer program and the non-transitory computer-readable storage medium are each computer program products containing the commands.

The claims, the figures and the description of the figures contain further features of embodiments of the present invention. The features and feature combinations mentioned above in the description, and the features and feature combinations mentioned below in the description of the figures and/or shown in the figures can be included by embodiments of the present invention not just in the particular combination stated but also in other combinations. In particular, the present invention can include embodiments and feature combinations that do not have all the features of a claim in the original wording. Furthermore, the present invention can include embodiments and feature combinations that go beyond or differ from the feature combinations presented in the dependency references of the claims.

### BRIEF DESCRIPTION OF THE DRAWINGS

The present invention is described in greater detail below with reference to specific exemplary embodiments and associated schematic drawings. In the figures, identical or functionally equivalent elements can be denoted by the same reference characters. The description of identical or functionally equivalent elements is not necessarily repeated when referring to different figures.

In the figures:

FIG. 1 shows a schematic representation of a medical apparatus and of an exemplary embodiment of a data processing apparatus according to the present invention; and

FIG. 2 shows a schematic flow diagram of an exemplary embodiment of a computer-implemented method according to the present invention for human-machine interaction for operating a medical apparatus.

### DETAILED DESCRIPTION

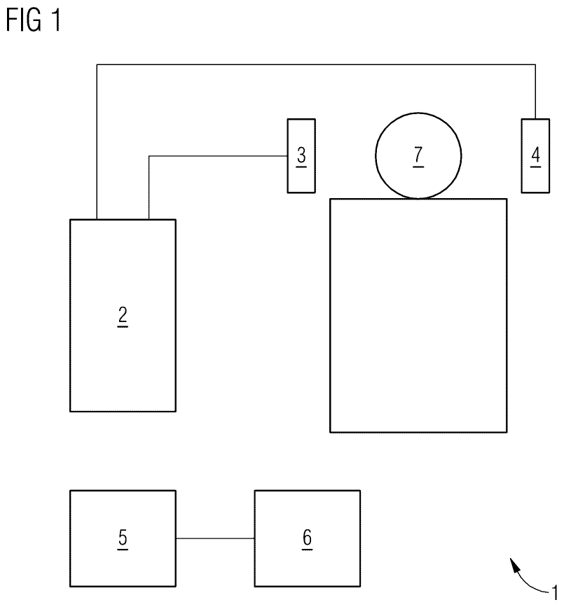

In FIG. 1 are a medical apparatus 1 and an exemplary embodiment of a data processing apparatus 5 according to the present invention. The data processing apparatus 5 is connected, for example, to a display device 6.

The medical apparatus 1 is shown purely by way of example and without loss of generality as an X-ray imaging system having an X-ray source 3, an X-ray detector 4 and a control unit 2. This can be used to perform X-ray imaging on a patient 7.

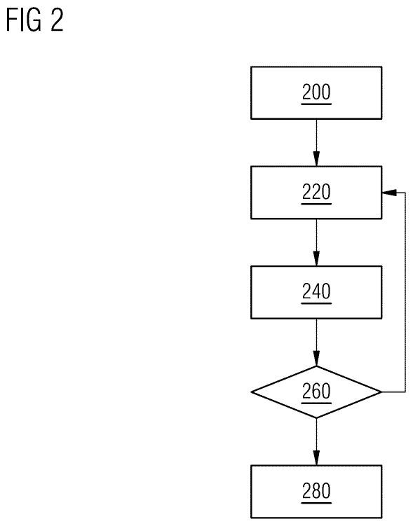

The data processing apparatus 5 has at least one computing unit, which is configured to perform a computer-implemented method, according to embodiments of the present invention, for human-machine interaction for operating the medical apparatus 1. FIG. 2 shows a schematic flow diagram of an exemplary embodiment of such a computer-implemented method.

In step 200, a first user input is captured, which specifies an intention of a user to operate the medical apparatus 1 for a particular usage case. In step 220, depending on the medical apparatus 1 and the usage case, at least one input suggestion is generated for a computer-implemented language model, and a representation of the at least one input suggestion is output, for example on the display device 6.

In step 240, a second user input is captured, which validates a first input suggestion of the at least one input suggestion or rejects the at least one input suggestion. In step 260, the second user input is analyzed. If the at least one input suggestion was rejected by the second user input, the method proceeds with step 220, for example, and once again at least one input suggestion is generated, and so on.

If the first input suggestion was validated by the second user input, then in step 280 user information for controlling the medical apparatus 1 for the usage case is generated and output, for instance on the display device 6, by applying the language model to the first input suggestion. The language model can be stored in the at least one computing unit and/or another computing unit, for example.

In some embodiments, the language model can be based on the large language model GPT-4, for example, which can process a plurality of languages, recognize images and capture extremely wide contextual input. In particular, it can handle various modalities such as text, image, video and audio. This model can be refined on the basis of specific medical publications, for example relating to the medical application of ultrasound, X-rays and so on, to radiology in general, to differential diagnosis or case history, and so on.

In various embodiments, the user can use natural language to instruct the system with a suspected diagnosis, whereupon the system can offer to the user different options, possibly also using alternative medical apparatuses, and can suggest the best possible solution within the capabilities of the medical apparatus 1. The user can be guided step by step by the user information, for example from the positioning of the patient 7, through the arrangement of certain components of the medical apparatus 1, for instance of the X-ray source 3, or, in other embodiments, of an ultrasound transducer, right up to the configuration of the medical apparatus 1, and so on.

Since the system knows, as it were, what to look for, in some embodiments it could also automatically set parameters of the medical apparatus 1, perform relevant measurements, and produce a report containing a suggested diagnosis, differential diagnoses to be taken into account, and/or follow-up examinations for refining the diagnosis. The user information can be provided with context-related literature references, so that the user can check the suggestions.

In particular, one or more embodiments of the present invention can reduce the risk of hallucinated responses by the language model.

To achieve this, the behavior of the language model can be adapted in particular for specific usage cases in the medical technology field by refining the language model using a dataset relevant to these fields of use. This can narrow down the knowledge of the language model in order to reduce the likelihood of producing irrelevant or hallucinated content.

The outputs from the language model can be filtered in a postprocessing step, for example, in order to filter out content that does not satisfy certain criteria, for instance because it does not originate from trustworthy sources or contains certain keywords.

An example of a dialog by the user with the system might look as follows:

User: “I want to use an ultrasound examination in order to confirm or rule out a cholecystectomy.”

Input suggestion: “Please explain in detail how a doctor uses an ultrasound imaging procedure to diagnose medical conditions that might require a cholecystectomy. Start with a checklist of the instruments required. Add information about the aim of the procedure, the steps it contains, and the way in which to interpret the ultrasound results in order to determine whether a cholecystectomy is needed. Add the settings for the ultrasound system; use only data from the validated library”.

After the user has validated the input suggestion, the language model can then produce the user information on the basis thereof.

An example of an input suggestion and corresponding user information might look as follows:

Input suggestion: “I would like you to act as a radiology expert and focus on imaging the liver. As I am a clinician, I do not need the fundamentals explained. I will describe a clinical case and a diagnostic impression, and I want you to suggest the available options for confirming the diagnosis, possible differential diagnoses and the examinations suitable for a final diagnosis. My case is: a 42-year old patient, born in Mexico, with a personal history of an appendectomy and diverticular disease, with an acute episode every two years in the last 8 years. His BMI is 27 and he drinks about 3-4 beers per week and occasionally a glass of whiskey. In a routine blood test, the lipase value appeared four times above the threshold value (no abdominal pain); transaminases and erythrocyte sedimentation rate were slightly elevated. Blood count, kidney function and biochemistry were normal.”

User information: “Based on the medical history and the laboratory results of the patient, I am assuming that you suspect possible liver disease. In this case, there are several options for further examinations, differential diagnoses and treatments.

Diagnostic options for confirming the diagnosis:
1. Ultrasound (US)—a liver ultrasound is a non-invasive, cost-effective, first-line imaging procedure that can be used to assess the size and echotexture of the liver and to identify focal lesions or diffuse anomalies.
2. Magnetic resonance tomography (MRT)—MRT using hepatobiliary contrast agent or liver-specific contrast agents can provide detailed information about the liver parenchyma and lesions. It can also help to assess the biliary tree.
3. Computed tomography (CT)—a CT examination using intravenous contrast agent can provide detailed information about the liver parenchyma, the vessels and possible lesions.

Possible differential diagnoses:
1. Nonalcoholic fatty liver disease (NAFLD)—given the BMI of the patient and the slightly elevated transaminases, NAFLD might be a possibility. This can develop into non-alcoholic steatohepatitis (NASH) and eventually into cirrhosis.
2. Alcoholic liver disease-even though the patient's alcohol consumption is only moderate, this could contribute to liver disease.
3. Hepatitis—viral hepatitis (A, B, C, D or E), autoimmune hepatitis or drug-induced hepatitis should be considered.
4. Primary sclerosing cholangitis (PSC)—PSC is a rare liver disease that can cause slightly elevated transaminases and lipases. It is more common in patients with inflammatory bowel disease, but can also occur in patients with diverticular disease.
5. Liver lesions—hemangiomas, focal nodular hyperplasia (FNH), hepatic adenomas or hepatocellular carcinoma (HCC) can be possible causes of liver abnormalities.

Examination to obtain a definitive diagnosis:
1. Serology—test for viral hepatitis markers (HAV, HBV, HCV, HDV, HEV), autoimmune markers (ANA, ASMA, LKM-1) and liver function tests (albumin, bilirubin, INR).
2. Imaging—start with an ultrasound examination of the liver to identify structural anomalies. Depending on the findings, further imaging using CT or MRT may be indicated.
3. Elastography—FibroScan or MR elastography can be used for non-invasive assessment of liver fibrosis and to determine further steps.
4. Liver biopsy—a liver biopsy can provide the definitive histological evidence in cases in which the diagnosis remains uncertain or advanced liver disease is suspected.
   Based on the results of the examination, a final diagnosis can be made and appropriate treatment and care put into place.”

It is also possible that the system provides a single comprehensive response, including, for example, the suggested system settings, the step-by-step procedure in text form and schematic diagrams for each step, and relevant clinical reasoning. In some embodiments, visual representations of the respective information sources can be displayed, for instance for each section of the response, with the particular sentence being highlighted visually so that a knowledgeable user can validate it directly. This is the key to facilitating clinical integration.

In some embodiments, it is also possible that medical personnel wear an AR headset, via which the system can provide information about how certain procedures are meant to be carried out. It may be the case, however, that the medical personnel do not have the necessary qualifications to validate the guidance. The validation can then be performed by a suitably qualified third party, who is in another location and, for example, looks after a plurality of groups accordingly. It would then be possible to make efficient use not only of the knowledge and experience of the third party but also of the resources of the medical personnel and the capability of the relevant technology.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A computer-implemented method for human-machine interaction for operating a medical apparatus, the computer-implemented method comprising:
capturing a first user input specifying an intention to operate the medical apparatus for a usage case;
generating, depending on the medical apparatus and the usage case, at least one input suggestion for a computer-implemented language model;
outputting a representation of the at least one input suggestion;
capturing a second user input that validates or rejects a first input suggestion of the at least one input suggestion; and
in response to validation of the first input suggestion by the second user input, generating and outputting user information for controlling the medical apparatus for the usage case by applying the computer-implemented language model to the first input suggestion.

2. The computer-implemented method as claimed in claim 1, wherein the user information includes at least one of
system settings for the medical apparatus,
guidance for operating the medical apparatus for the usage case, or
one or more pictorial representations of a procedure for operating the medical apparatus for the usage case.

3. The computer-implemented method as claimed in claim 1, wherein the computer-implemented language model includes a trained artificial neural network.

4. The computer-implemented method as claimed in claim 3, wherein the trained artificial neural network is a generative pre-trained transformer.

5. The computer-implemented method as claimed in claim 1, wherein
the at least one input suggestion includes the first input suggestion and a second input suggestion, and
the second user input either selects and validates the first input suggestion or rejects the at least one input suggestion.

6. The computer-implemented method as claimed in claim 1, further comprising:
outputting source information relating to the user information.

7. The computer-implemented method as claimed in claim 6, wherein
the source information includes at least one of a textual or pictorial representation of at least one extract from an information source for the user information, and
the computer-implemented method includes displaying the at least one of the textual or pictorial representation on a display device, wherein
a part of the at least one of the textual or pictorial representation that is relevant to the user information is highlighted visually.

8. The computer-implemented method as claimed in claim 1, wherein, upon rejection of the at least one input suggestion by the second user input, the computer-implemented method further comprises:
generating, depending on the medical apparatus and the usage case, at least one further input suggestion for the computer-implemented language model;
outputting a representation of the at least one further input suggestion;
capturing a third user input that validates or rejects a first further input suggestion of the at least one further input suggestion; and
in response to validation of the first further input suggestion by the third user input, generating and outputting further user information for controlling the medical apparatus for the usage case by applying the computer-implemented language model to the first further input suggestion.

9. The computer-implemented method as claimed in claim 8, wherein the at least one further input suggestion is generated according to content of the second user input.

10. The computer-implemented method as claimed in claim 1, further comprising:
capturing a fourth user input that specifies at least one of an acceptance or applicability of the user information for the usage case; and
in response to the fourth user input specifying that the user information was at least one of not accepted or not applicable, generating and outputting further user information for controlling the medical apparatus for the usage case by applying the computer-implemented language model to the first input suggestion and the fourth user input.

11. The computer-implemented method as claimed in claim 1, wherein the medical apparatus is an imaging modality.

12. The computer-implemented method as claimed in claim 1, further comprising:
in response to validation of the first input suggestion by the second user input, at least one of automatically setting a parameter of the medical apparatus on the basis of the user information, or automatically controlling the medical apparatus on the basis of the user information to perform a measurement automatically.

13. A data processing apparatus having at least one computing unit, the at least one computing unit configured to perform the computer-implemented method as claimed in claim 1.

14. A non-transitory computer-readable storage medium storing computer-executable instructions that, when executed by a data processing apparatus, cause the data processing apparatus to perform the computer-implemented method as claimed in claim 1.

15. The computer-implemented method as claimed in claim 5, further comprising:
outputting source information relating to the user information.

16. The computer-implemented method as claimed in claim 15, wherein
the source information includes at least one of a textual or pictorial representation of at least one extract from an information source for the user information, and
the computer-implemented method includes displaying the at least one of the textual or pictorial representation on a display device, wherein
a part of the at least one of the textual or pictorial representation that is relevant to the user information is highlighted visually.

17. The computer-implemented method as claimed in claim 16, wherein, upon rejection of the at least one input suggestion by the second user input, the computer-implemented method further comprises:
generating, depending on the medical apparatus and the usage case, at least one further input suggestion for the computer-implemented language model;
outputting a representation of the at least one further input suggestion;
capturing a third user input that validates or rejects a first further input suggestion of the at least one further input suggestion; and
in response to validation of the first further input suggestion by the third user input, generating and outputting further user information for controlling the medical apparatus for the usage case by applying the computer-implemented language model to the first further input suggestion.

18. The computer-implemented method as claimed in claim 7, wherein, upon rejection of the at least one input suggestion by the second user input, the computer-implemented method further comprises:
generating, depending on the medical apparatus and the usage case, at least one further input suggestion for the computer-implemented language model;
outputting a representation of the at least one further input suggestion;
capturing a third user input that validates or rejects a first further input suggestion of the at least one further input suggestion; and
in response to validation of the first further input suggestion by the third user input, generating and outputting further user information for controlling the medical apparatus for the usage case by applying the computer-implemented language model to the first further input suggestion.

19. The computer-implemented method as claimed in claim 8, further comprising:
capturing a fourth user input that specifies at least one of an acceptance or applicability of the user information for the usage case; and
in response to the fourth user input specifying that the user information was at least one of not accepted or not applicable, generating and outputting further user information for controlling the medical apparatus for the usage case by applying the computer-implemented language model to the first input suggestion and the fourth user input.

20. The computer-implemented method as claimed in claim 8, further comprising:
in response to validation of the first input suggestion by the second user input, at least one of automatically setting a parameter of the medical apparatus on the basis of the user information, or automatically controlling the medical apparatus on the basis of the user information to perform a measurement automatically.
